(19) United States
(12) Patent Application Publication
(10) Pub. No.: US 2025/0314564 A1
(43) Pub. Date: Oct. 9, 2025

(54) APPARATUS, SYSTEM, AND METHOD FOR ODOR ASSESSMENT

(71) Applicant: Steams, Conrad and Schmidt, Consulting Engineers, Inc., Reston, VA (US)

(72) Inventors: Paul SCHAFER, Ogden, UT (US); David HOSTETTER, Lancaster, PA (US); Joshua SCHMIDT, Santa Maria, CA (US); Sam DEAN, Manheim, PA (US); Sam RICE, Lancaster, PA (US); Gavin MARKLEY, Augusta, KS (US); Jordan HUNT, Gillette, WY (US); Sean GORDON, Fair Oaks, CA (US); Phil CARRILLO, Huntington Beach, CA (US)

(73) Assignee: Stearns, Conrad and Schmidt, Consulting Engineers, Inc., Reston, VA (US)

(21) Appl. No.: 19/169,253

(22) Filed: Apr. 3, 2025

Related U.S. Application Data
(60) Provisional application No. 63/573,656, filed on Apr. 3, 2024.

(51) Int. Cl. G01N 1/22; G01N 1/24

(52) U.S. Cl. CPC G01N 1/2202; G01N 1/24

(57) ABSTRACT

A system, apparatus, and method for odor assessment. Trace levels of sulfur dioxide (SO₂) are sampled and analyzed in combination with the thermal oxidation of the sample stream. Such analyses are used as a surrogate for odors present in cannabis growing facilities instead of measuring terpene concentrations to determine the presence of objectionable odors. A cloud-based remote monitoring and control system may provide for remote monitoring, diagnostics, live data viewing, visualization, graphing, historical data downloading, reporting, alarms, and data analytics.

### CROSS-REFERENCE TO RELATED APPLICATIONS

This application claims priority to U.S. Provisional Application 63/573,656, filed Apr. 3, 2024, the entire contents of which are hereby incorporated by reference.

### BACKGROUND

Odor assessments, for example, in greenhouses, cultivation areas, and processing areas, are difficult and costly. With respect to cannabis cultivation and processing, most odor assessments have focused on terpene concentrations. However, a more cost-effective solution that provides a better correlation to objectionable odors is desired.

Furthermore, sulfonated compounds are common causes of objectionable odors resulting from emissions from a multitude of source types. However, there is no method available to speciate the relative compounds in an affordable and real-time basis.

### SUMMARY

According to at least one exemplary embodiment, a system for odor assessment is disclosed. The system includes remote monitoring and control software and at least one measuring apparatus in communication with the remote monitoring and control software. The measuring apparatus includes a thermal oxidizer adapted to scrub ambient SO₂ from a sample and to convert sulfonated compounds in the sample to SO₂, and a trace level analyzer in fluid communication with the thermal oxidizer and adapted to detect total reduced sulfur (TRS) concentrations in a gas stream flowing out from the thermal oxidizer. The system for odor assessment is configured to determine a presence of objectionable odors by using the detected TRS concentrations as a surrogate for the presence of objectionable odors.

The remote monitoring and control software is configured to view, analyze, and record live data gathered from the at least one measuring apparatus, view and analyze recorded historical data, configure operational parameters of the system for odor assessment, and provide for remote monitoring, diagnostics, live data viewing, visualization, graphing, historical data downloading, reporting, alarms, and data analytics of the system for odor assessment.

According to another exemplary embodiment, a measuring apparatus for odor assessment is disclosed. The measuring apparatus includes a thermal oxidizer adapted to scrub ambient SO2 from a sample, and to convert sulfonated compounds in the sample to SO2, and a trace level analyzer in fluid communication with the thermal oxidizer and adapted to detect TRS concentrations in a gas stream flowing out from the thermal oxidizer. The measuring apparatus is configured to determine a presence of objectionable odors by using the detected TRS concentrations as a surrogate for the presence of objectionable odors. The measuring apparatus can further include at least one sample intake line in fluid communication with a manifold, at least one valve, the valve being disposed between the at least one sample intake line and the manifold, a pump in fluid communication with the manifold and the thermal oxidizer, and at least one exhaust line in fluid communication with the trace level analyzer. The measuring apparatus can further include a mobile cabinet enclosing components of the measuring apparatus, wherein the at least one sample intake line is in fluid communication with an environment external to the apparatus, or wherein a plurality of intake lines are in fluid communication with different areas of an environment external to the apparatus. The measuring apparatus can further include a controller adapted to control the operation of the at least one valve, the pump, the thermal oxidizer, and the trace level analyzer, receive and record data from the thermal oxidizer and the trace level analyzer, and communicate with the remote monitoring and control software.

According to another exemplary embodiment, a method for odor assessment is disclosed. The method can include the steps of providing a sample stream to a thermal oxidizer, scrubbing ambient SO2 from the sample stream, converting sulfonated compounds in the sample stream to SO2, flowing the sample stream to a trace level analyzer, detecting TRS concentrations in the sample stream, and determining a presence of objectionable odors by using the detected TRS concentrations as a surrogate for the presence of objectionable odors. The method can further include the steps of recording TRS concentration data, correlating the TRS concentration data to environmental factors of the location where the TRS concentration data was obtained, developing correlation factors of the TRS concentration data and odors specific to the location based on the TRS concentration data and the environmental factors. The method can further include the steps of predicting odor levels for the location based on the correlation factors and determining potential odor impacts in real time by comparing odor levels for the location to defined benchmarks.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

FIG. 1 shows an exemplary embodiment of a remote monitoring and control system.

FIG. 2 diagrammatically shows an exemplary embodiment of a mobile scent analyzer.

FIG. 3A is a perspective view of an exemplary embodiment of a mobile scent analyzer.

FIG. 3B is a front view of an exemplary embodiment of a mobile scent analyzer.

FIG. 3C is another front view of an exemplary embodiment of a mobile scent analyzer.

FIG. 4 is a sample flow diagram of an exemplary embodiment of a mobile scent analyzer.

FIG. 5 is an exemplary interface of a remote monitoring and control system.

FIG. 6 is another exemplary interface of a remote monitoring and control system.

FIG. 7 is another exemplary interface of a remote monitoring and control system.

FIG. 8 is another exemplary interface of a remote monitoring and control system.

FIG. 9 is another exemplary interface of a remote monitoring and control system.

FIG. 10 is another exemplary interface of a remote monitoring and control system.

FIG. 11 is another exemplary interface of a remote monitoring and control system.

FIG. 12 is another exemplary interface of a remote monitoring and control system.

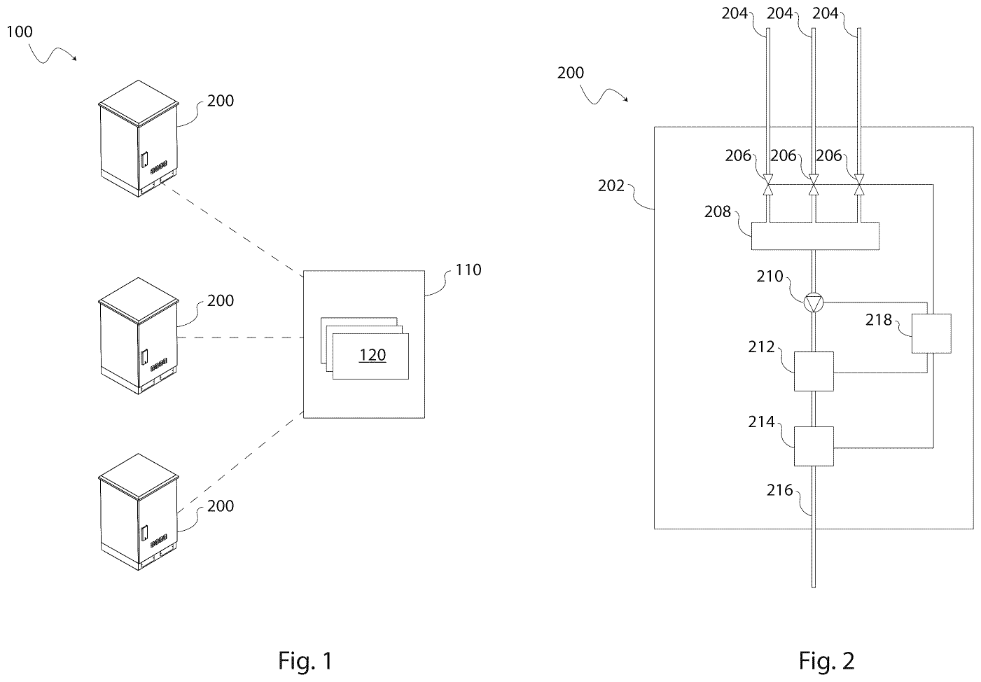

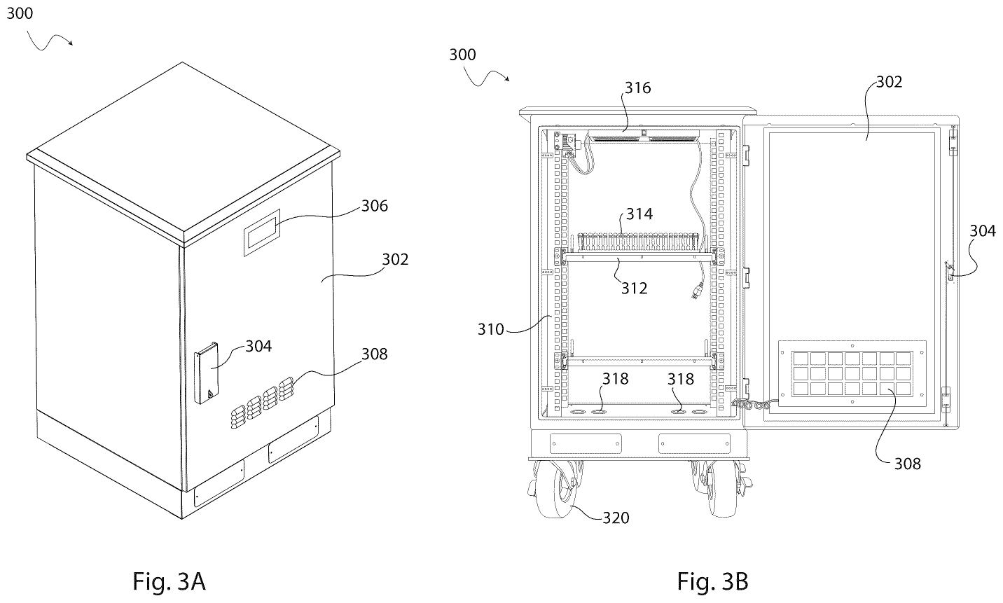

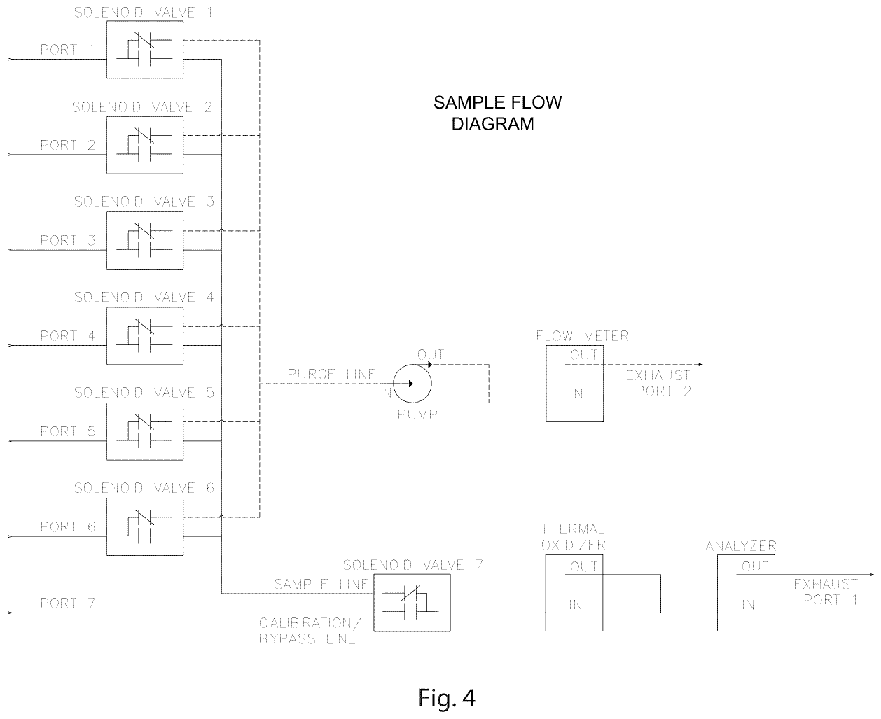

### DETAILED DESCRIPTION

Aspects of the invention are disclosed in the following description and related drawings directed to specific embodiments of the invention. Those skilled in the art will recognize that alternate embodiments may be devised without departing from the spirit or the scope of the claims. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiment are not necessarily to be construed as preferred or advantageous over other embodiments. Moreover, the terms “embodiments of the invention”, “embodiments” or “invention” do not require that all embodiments of the invention include the discussed feature, advantage or mode of operation.

Further, many of the embodiments described herein may be described in terms of sequences of actions to be performed by, for example, elements of a computing device. It should be recognized by those skilled in the art that the various sequence of actions described herein can be performed by specific circuits (e.g., application specific integrated circuits (ASICs)) and/or by program instructions executed by at least one processor. Additionally, the sequence of actions described herein can be embodied entirely within any form of computer-readable storage medium such that execution of the sequence of actions enables the processor to perform the functionality described herein. Thus, the various aspects of the present invention may be embodied in a number of different forms, all of which have been contemplated to be within the scope of the claimed subject matter. In addition, for each of the embodiments described herein, the corresponding form of any such embodiments may be described herein as, for example, “a computer configured to” perform the described action.

Embodiments disclosed herein may include a mobile scent analyzer for cannabis growing facilities such as greenhouses, cultivation areas, and processing areas. The embodiments may be adapted to sample and analyze trace levels of sulfur dioxide (SO₂) in combination with thermal oxidation of the sample stream. The embodiments may utilize these analyses as a surrogate for odors present in the cannabis growing facilities instead of measuring terpene concentrations to determine the presence of objectionable odors.

Embodiments disclosed herein may also include a cloud-based remote monitoring and control system that allows for remote monitoring, diagnostics, live data viewing, visualization, graphing, historical data downloading, reporting, alarms, and data analytics.

According to the exemplary embodiments, the physical structure of the mobile scent analyzer may include a wheeled cabinet, within which may be disposed a thermal oxidizer for converting all sulfur to SO₂ and a trace level SO₂ analyzer for analyzing the resulting gas stream for SO₂. The thermal oxidizer may be in communication with the external environment via sample lines. Interposed between the sample lines and the oxidizer may be one or more solenoid valves for opening and closing the sample lines, a sample pump for drawing in the sample, and a sample manifold connecting the valves to the pump. After analysis by the SO₂ analyzer, the sampled gas may be exhausted to the external environment via an exhaust. A programmable logic controller may control the operation of the above components, receive data from the analyzer, and communicate with a human-machine interface and the cloud system.

The cloud-based remote monitoring and control system may allow for remote monitoring, diagnostics, live data viewing, visualization, graphing, historical data downloading, reporting, alarms, and data analytics, which may be observed and controlled via interfaces of the remote software of the system. In addition, the user can remotely select monitoring locations and perform diagnostics on the system via interfaces of the software. The user can also remotely control the operations of the system via the interfaces.

Embodiments disclosed herein may facilitate providing general assessments of total reduced sulfur (TRS) concentrations at cannabis facilities, asphalt plants, landfills, and other facilities. Embodiments disclosed herein may further facilitate assessing the relative effectiveness of odor control methods and specific odor control equipment. Embodiments disclosed herein may further facilitate assessing low-level (parts-per-billion) total reduced sulfur concentrations for the purpose of odor gradient assessment in cultivation facilities. Embodiments disclosed herein may further facilitate assessing emission rates and dynamics through vented greenhouses. Embodiments disclosed herein may further facilitate eliminating or reducing the need for odor panels and shipping of samples for odor analysis.

Embodiments of the trace level TRS monitoring system, with the collection of discrete odor samples, can allow for the development of correlation factors of TRS measurement to odors specific to the source type being assessed. Embodiments of the system can then predict odor levels based on the correlation factors and compare the odor levels to defined benchmarks to arrive at a real time assessment of potential odor impacts.

According to at least one exemplary embodiment, an apparatus, system, and method for odor assessment is disclosed. A system for odor assessment 100 may include at least one measuring apparatus 200, which may be in communication with a remote monitoring and control software 110. Remote monitoring and control software 110 can provide for remote live data viewing, visualization, graphing, historical data downloading, automatic reporting, alarming, and data analytics. Furthermore, the remote monitoring and control software 110 can allow a user to remotely select monitoring locations and perform diagnostics on elements of system 100 via an interactive dashboard provided by an interface 120 of software 110. The user can also remotely control the operation of system 100 via interfaces 120 provided by software 110.

Embodiments of the measuring apparatus can include an eclosure 202, for example a mobile cabinet, within which may be disposed a thermal oxidizer for converting sulfur to SO₂ and a trace level SO₂ analyzer for analyzing the resulting gas stream for SO₂. The thermal oxidizer may be in communication with the external environment via sample lines. Interposed between the sample lines and the oxidizer may be a solenoid valve for opening and closing the sample lines, a sample pump for drawing in the sample, and a sample manifold connecting the valves to the pump. After analysis by the SO₂ analyzer, the sampled gas may be exhausted to the external environment via an exhaust. A programmable logic controller may control the operation of the components of the measuring apparatus, may receive data from the analyzer, and may communicate with a human-machine interface and software 110.

The manifold and valve arrangement can provide for the analysis of gases at multiple locations or test points within a subject area in which the measuring apparatus is present. The measuring apparatus may also be modified to measure additional meteorological and or chemical concentration parameters. The analysis of total reduced sulfur concentrations may act as a surrogate for odor levels within or at a known odor source. In some exemplary embodiments, the measurement of total reduced sulfur concentrations may be used as a surrogate measurement for odor measurements within various facilities, for example, such as cannabis greenhouses, cultivation areas, cannabis processing areas, and so forth.

Using embodiments of the trace level TRS monitoring system as disclosed herein, along with the collection of discrete odor samples, can allow for development of correlation factors of the TRS measurement to odors specific to the source type being assessed. Embodiments of the system can then predict odor levels and compare the odor levels to defined benchmarks for a real time assessment of potential odor impacts.

FIG. 2 shows an exemplary embodiment of a measuring apparatus 200, for example, a mobile scent analyzer. Sample intake lines 204 may be provided for communication between the interior of the enclosure 202 and the surrounding environment. A plurality of intake lines 204 may be provided, so as to allow a single measuring apparatus to obtain samples from different locations. The sample intake lines 204 may enter the measuring apparatus via the intake ports. The distal ends of the sample intake lines 204 may be positioned in various areas of the surrounding environment from which air samples are desired to be taken. The internal ends of each sample intake line 204 may be in communication with a valve 206, for example a solenoid valve. The valve 206 may be adapted to selectively open and close a particular sample line when it is desired to sample the air of the area in which the distal end of the particular sample line is located. A plurality of valves 206 may be provided, with each valve 206 corresponding to a sample intake line 204. The plurality of valves 206 may further be in communication with a manifold 208, which in turn may be in communication with a pump 210. The pump 210 may be adapted to intake sample air through the sample lines 204, open valves 206, and manifold 208, and direct the sample air towards a thermal oxidizer 212.

Initially, the thermal oxidizer 212 may scrub any ambient SO₂ from the sample. The thermal oxidizer 212 may then convert any present sulfonated compounds in the sample air to SO₂. The air having sulfonated compounds oxidized to SO₂ may then be directed towards an SO₂ analyzer 214 that is coupled to the thermal oxidizer 212. The SO₂ analyzer 214 may detect any SO₂ that is present in the gas stream that flows out from the thermal oxidizer. In other words, the system essentially becomes a sulfur atom counter. Subsequent to analysis, the gas stream may flow out of the analyzer 214 and out of the measuring apparatus 200 via one or more exhaust lines 216.

The components of the TRS measuring apparatus 200 may be controlled by a programmable logic controller 218. The controller 218 may selectively activate and deactivate the valves and set a duration for activation of each valve. The controller 218 may also activate and deactivate the pump, thermal oxidizer, and analyzer as necessary for the operation described herein. The controller 218 may further receive and record data from the analyzer and thermal oxidizer. In an embodiment, the controller 218 may record sample data from the analyzer for a particular location when the analyzed data is within a user-adjustable deadband of recently sampled data in that location. The controller 218 may further transmit data to the remote monitoring and control software 110, which may include human-machine interfaces 120.

The measuring apparatus 200 can be provided as a cabinet 300 enclosing the components of the measuring apparatus. The cabinet may be provided with a hinged door 302 on which a locking mechanism 304, control panel 306, and vents 308 may be disposed. In the internal cavity of cabinet 300 may also be disposed an adjustable shelving arrangement 310 with slidable shelves 312 and rack 314, a plurality of fans 316 to provide positive or negative air pressure as desired, and a plurality of air intake and exhaust ports 318, through which the sample lines and the exhaust line may pass. The control panel 306 may facilitate control of the internal components of cabinet 300. Wheels 320 may be provided on the exterior of cabinet 300 to allow for ease of relocation of the cabinet.

Interfaces 120 of the remote monitoring and control software can present various observational and control abilities to a user of the system. For example, a user of the system may view and analyze live data gathered from one or more TRS measuring apparatuses, as well as view and analyze recorded historical data. The user may further utilize the software 110 to control and modify the operations of the system 100. As a non-limiting example, various operational parameters of the system 100 may be changed, including a number of zones where odor measurements are performed, active and inactive zones, zone identifications, and alarm setpoints.

The cloud-based remote monitoring and control system 100 may allow for remote monitoring, diagnostics, live data viewing, visualization, graphing, historical data downloading, reporting, alarms, and data analytics, which may be observed and controlled via interfaces 120 of the remote software 110 of the system 100. In addition, the user can remotely select monitoring locations and perform diagnostics on the system via interfaces of the software. The user can also remotely control the operations of the system via the interfaces.

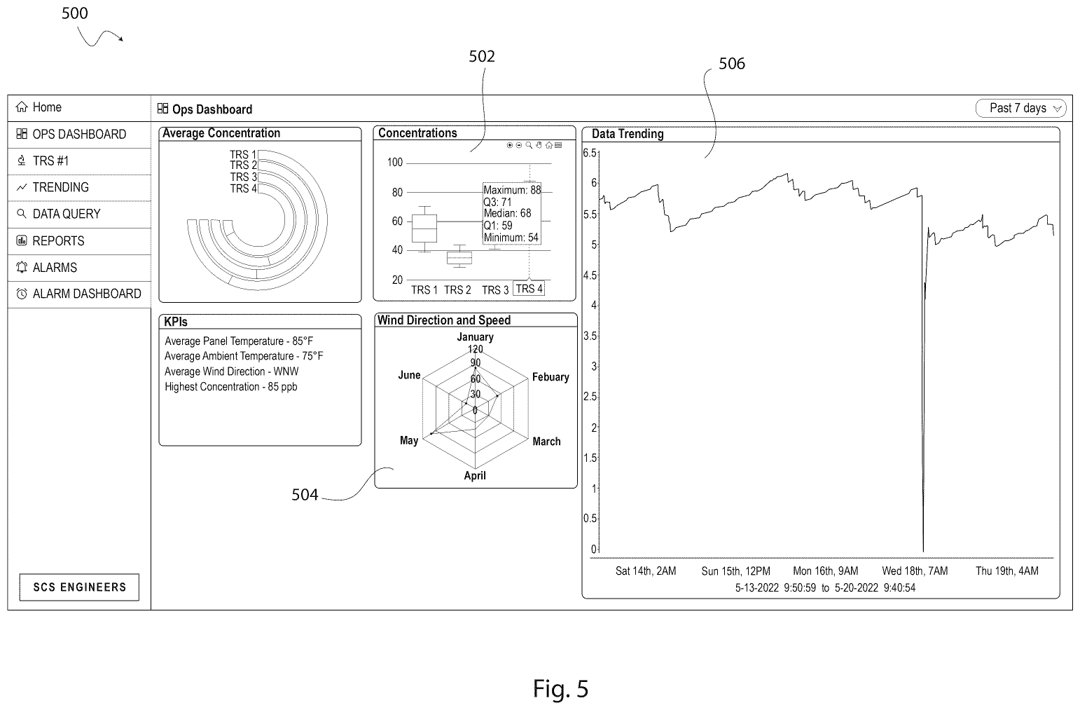

FIG. 5 shows an exemplary interface 500 showing conditions at several testing sites. Total reduced sulfur concentrations 502 are shown, as well as environmental conditions 504 such as wind speed, direction, and temperature. A historical graph 506 of measurements is also shown.

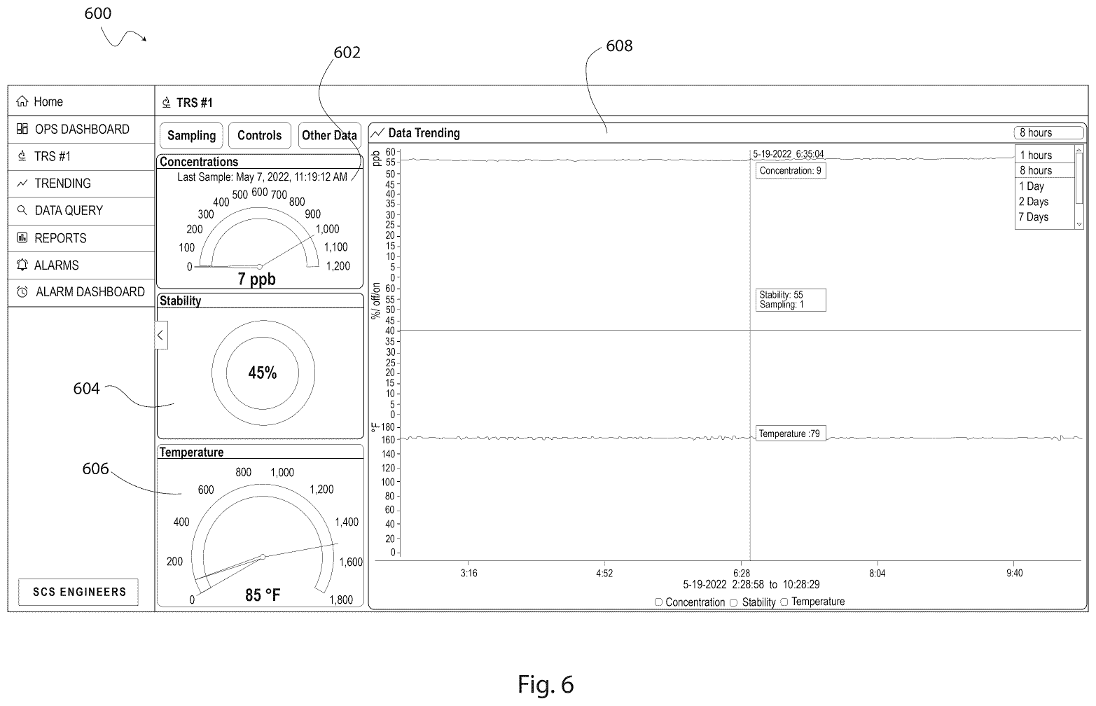

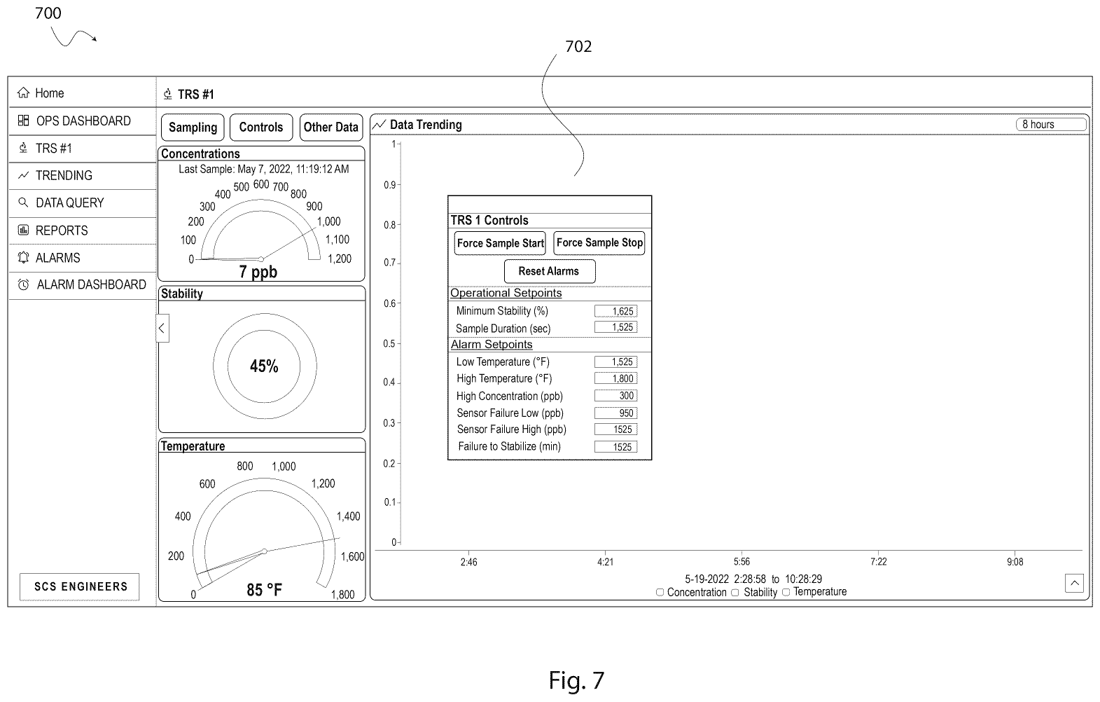

FIG. 6 shows an exemplary interface 600 showing current and historical conditions at a testing site 600. Total reduced sulfur concentrations 602 are shown, as well as measurement stability 604, ambient temperature 606, and historical data 608. FIG. 7 shows an exemplary interface 700 showing operational and alarm setpoints 702 at a testing site.

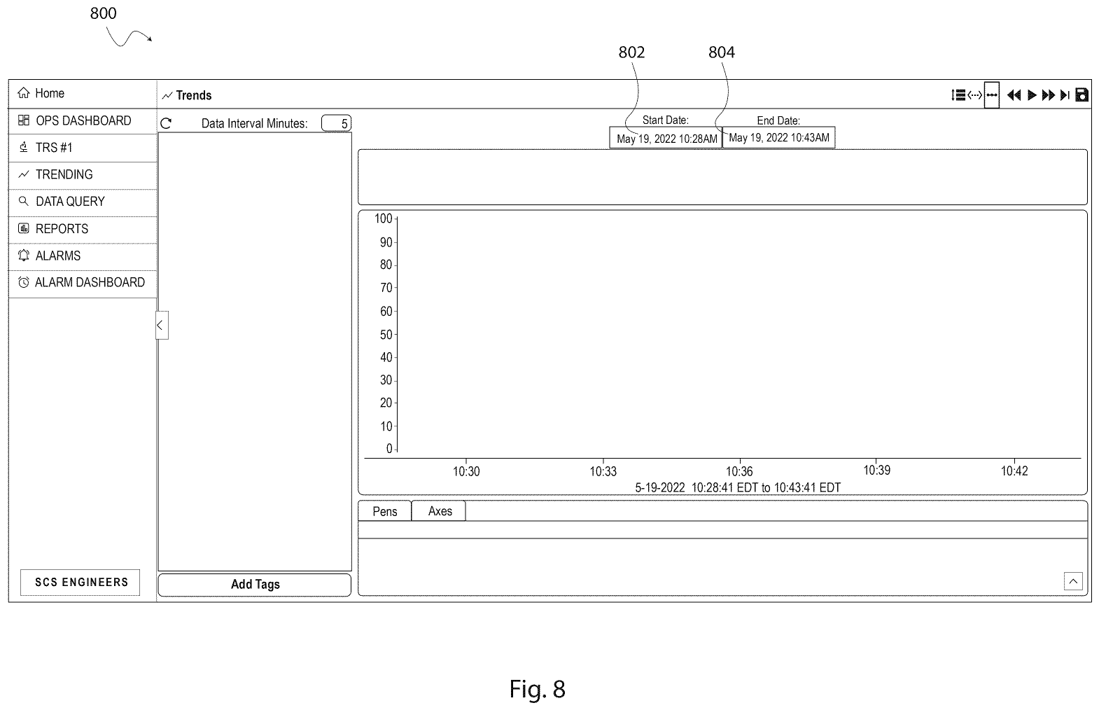

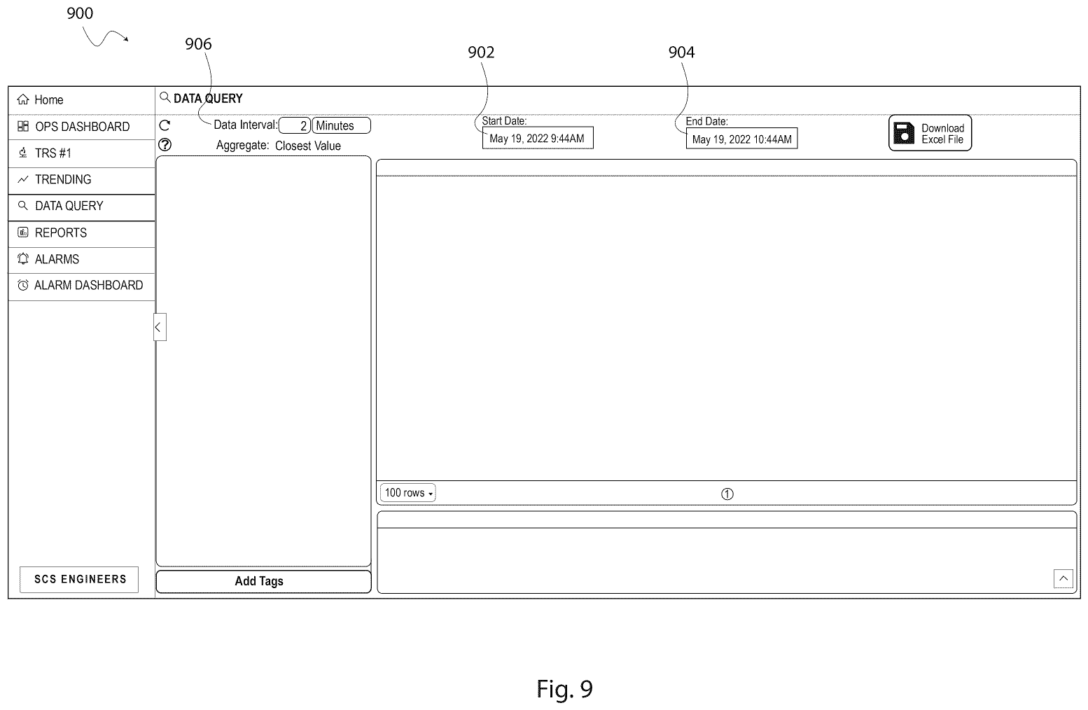

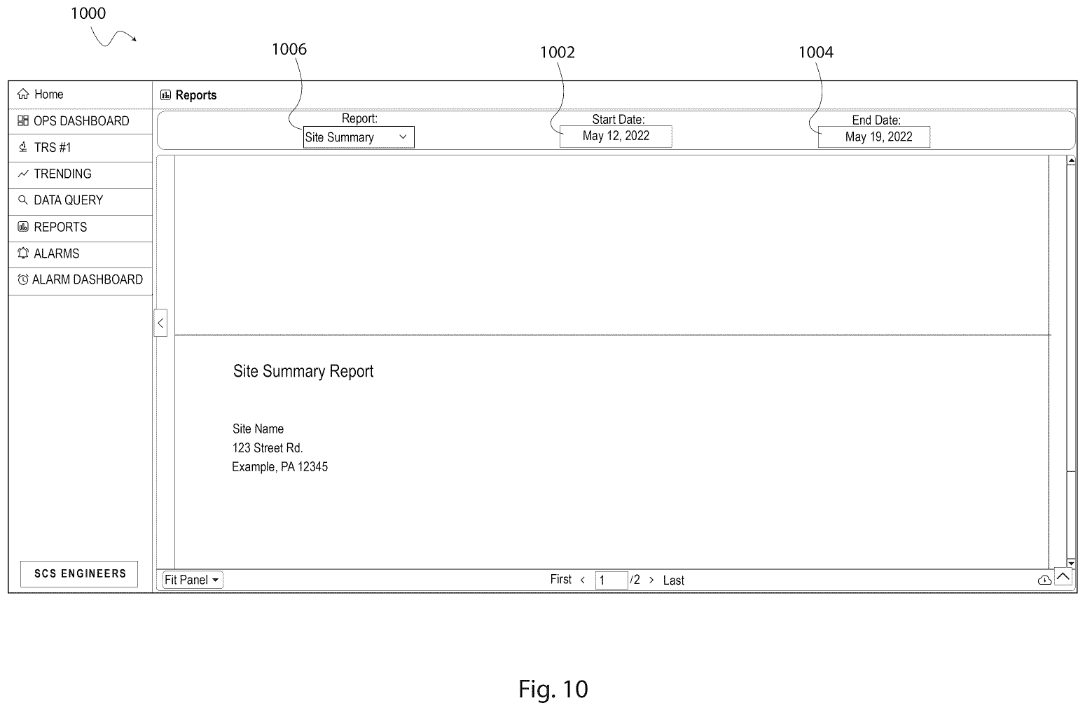

FIG. 8 shows an exemplary interface 800 for data sampling and generating trends for one or more testing sites. Start times 802, end times 804, and intervals for sampling 806 may be set. FIG. 9 shows an exemplary data query interface 900 for one or more testing sites. Start times 902, end times 904, and intervals for querying 906 may be set. FIG. 10 shows an exemplary report generation interface 1000 for one or more testing sites. Start times 1002, end times 1004, and report types 1006 may be set.

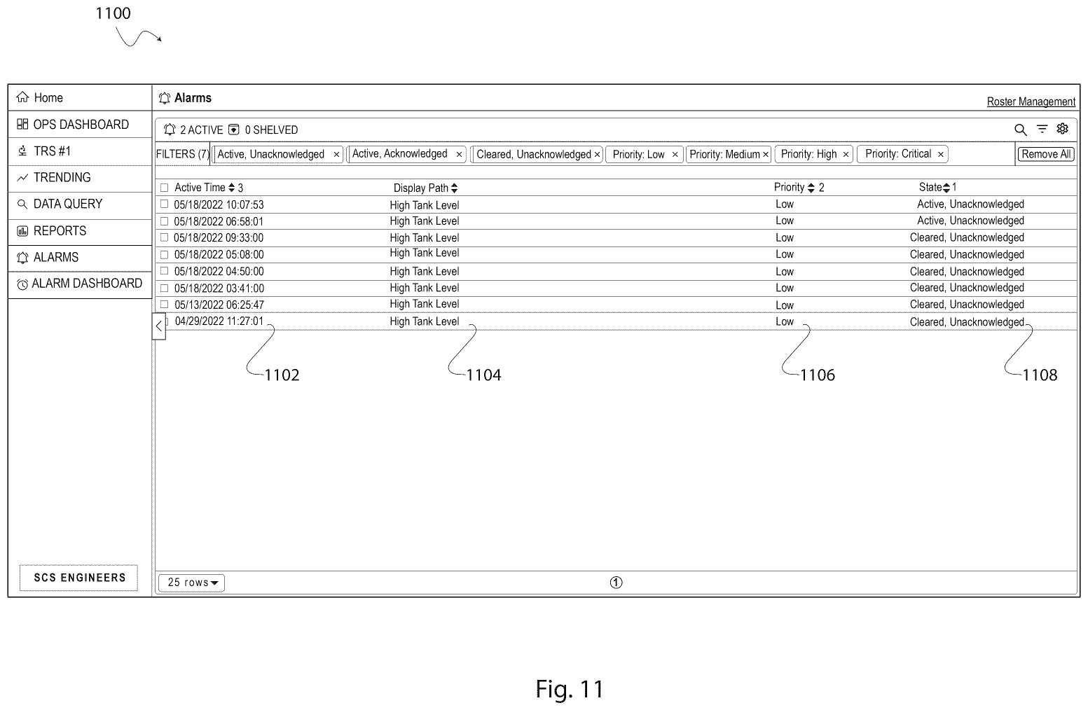

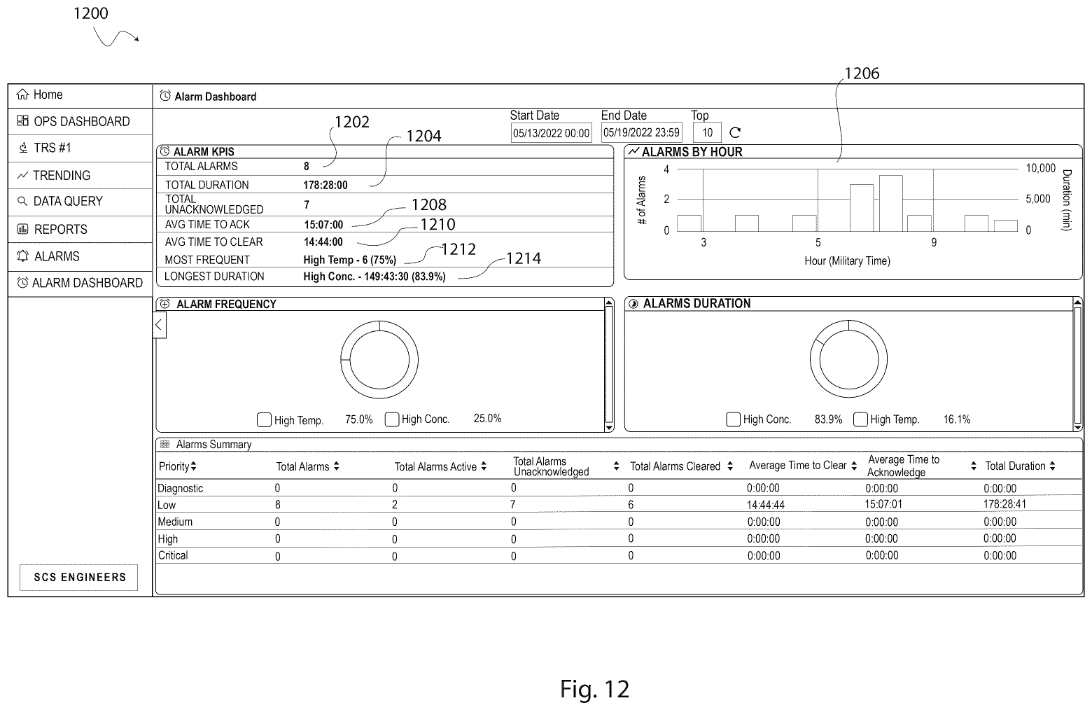

FIG. 11 shows an exemplary interface for alarm display 1100 for one or more testing sites. The time and date 1102, type of alarm 1104, priority 1106, and status 1108 may be shown. FIG. 12 shows an exemplary detailed alarm display 1200 for one or more testing sites. The total alarms 1202, duration of alarms 1204, time of alarms 1206, and acknowledgment time 1208 and clearing time of alarms 1210 may be shown. Additionally, the most frequent type of alarm 1212 and longest duration type of alarm 1214 may be shown.

The foregoing description and accompanying figures illustrate the principles, preferred embodiments, and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A system for odor assessment, comprising:
remote monitoring and control software; and
at least one measuring apparatus in communication with the remote monitoring and control software, the measuring apparatus comprising a thermal oxidizer adapted to scrub ambient SO₂ from a sample and to convert sulfonated compounds in the sample to SO₂, and a trace level analyzer in fluid communication with the thermal oxidizer and adapted to detect total reduced sulfur (TRS) concentrations in a gas stream flowing out from the thermal oxidizer;
wherein the system for odor assessment is configured to determine a presence of objectionable odors by using the detected TRS concentrations as a surrogate for the presence of objectionable odors.

2. The system of claim 1, the measuring apparatus further comprising:
at least one sample intake line in fluid communication with a manifold;
at least one valve, the valve being disposed between the at least one sample intake line and the manifold;
a pump in fluid communication with the manifold and the thermal oxidizer; and
at least one exhaust line in fluid communication with the trace level analyzer.

3. The system of claim 2, the measuring apparatus further comprising:
a plurality of sample intake lines, each sample intake line being in fluid communication with the manifold;
a plurality of valves, each valve being disposed between a corresponding sample intake line and the manifold.

4. The system of claim 2, the measuring apparatus further comprising a mobile cabinet enclosing components of the measuring apparatus, wherein the at least one sample intake line is in fluid communication with an environment external to the apparatus.

5. The system of claim 3, the measuring apparatus further comprising a mobile cabinet enclosing components of the measuring apparatus, wherein the plurality of intake lines are in fluid communication with different areas of an environment external to the apparatus.

6. The system of claim 2, the measuring apparatus further comprising a controller adapted to:
control the operation of the at least one valve, the pump, the thermal oxidizer, and the trace level analyzer;
receive and record data from the thermal oxidizer and the trace level analyzer; and
communicate with the remote monitoring and control software.

7. The system of claim 1, wherein the remote monitoring and control software is configured to:
view, analyze, and record live data gathered from the at least one measuring apparatus;
view and analyze recorded historical data;
configure operational parameters of the system for odor assessment; and
provide for remote monitoring, diagnostics, live data viewing, visualization, graphing, historical data downloading, reporting, alarms, and data analytics of the system for odor assessment.

8. The system of claim 7, further comprising a plurality of measuring apparatuses in communication with the remote monitoring and control software.

9. A measuring apparatus for odor assessment, comprising:
a thermal oxidizer adapted to scrub ambient SO₂ from a sample, and to convert sulfonated compounds in the sample to SO₂; and
a trace level analyzer in fluid communication with the thermal oxidizer and adapted to detect total reduced sulfur (TRS) concentrations in a gas stream flowing out from the thermal oxidizer;
wherein the measuring apparatus for odor assessment is configured to determine a presence of objectionable odors by using the detected TRS concentrations as a surrogate for the presence of objectionable odors.

10. The measuring apparatus of claim 9, further comprising:
at least one sample intake line in fluid communication with a manifold;
at least one valve, the valve being disposed between the at least one sample intake line and the manifold;
a pump in fluid communication with the manifold and the thermal oxidizer; and
at least one exhaust line in fluid communication with the trace level analyzer.

11. The measuring apparatus of claim 10, further comprising:
a plurality of sample intake lines, each sample intake line being in fluid communication with the manifold;
a plurality of valves, each valve being disposed between a corresponding sample intake line and the manifold.

12. The measuring apparatus of claim 10, further comprising:
a mobile cabinet enclosing components of the measuring apparatus;
wherein the at least one sample intake line is in fluid communication with an environment external to the apparatus.

13. The measuring apparatus of claim 11, further comprising:
a mobile cabinet enclosing components of the measuring apparatus;
wherein the plurality of intake lines are in fluid communication with different areas of an environment external to the apparatus.

14. The measuring apparatus of claim 10, further comprising a controller adapted to:
control the operation of the at least one valve, the pump, the thermal oxidizer, and the trace level analyzer;
receive and record data from the thermal oxidizer and the trace level analyzer; and
communicate with a remote monitoring and control software.

15. The measuring apparatus of claim 14, wherein the remote monitoring and control software is configured to:
view, analyze, and record live data gathered from the measuring apparatus;
view and analyze recorded historical data;
configure operational parameters of the measuring apparatus; and
provide for remote monitoring, diagnostics, live data viewing, visualization, graphing, historical data downloading, reporting, alarms, and data analytics.

16. The measuring apparatus of claim 14, wherein the remote monitoring and control software is configured to:
view, analyze, and record live data gathered from a plurality of measuring apparatuses;
view and analyze recorded historical data;
configure operational parameters of the plurality of measuring apparatuses; and
provide for remote monitoring, diagnostics, live data viewing, visualization, graphing, historical data downloading, reporting, alarms, and data analytics.

17. A method for odor assessment, comprising:
providing a sample stream to a thermal oxidizer;
scrubbing ambient SO₂ from the sample stream;
converting sulfonated compounds in the sample stream to SO₂;
flowing the sample stream to a trace level analyzer;
detecting total reduced sulfur (TRS) concentrations in the sample stream; and
determining a presence of objectionable odors by using the detected TRS concentrations as a surrogate for the presence of objectionable odors.

18. The method of claim 17, further comprising:
recording TRS concentration data;
correlating the TRS concentration data to environmental factors of the location where the TRS concentration data was obtained;
developing correlation factors of the TRS concentration data and odors specific to the location based on the TRS concentration data and the environmental factors.

19. The method of claim 18, further comprising predicting odor levels for the location based on the correlation factors.

20. The method of claim 18, further comprising determining potential odor impacts in real time by comparing odor levels for the location to defined benchmarks.
